(19) United States
(12) Patent Application Publication
(10) Pub. No.: US 2022/0062136 A1
(43) Pub. Date: Mar. 3, 2022

(54) COMPOSITIONS AND METHODS FOR CLEANSING KERATIN MATERIALS

(71) Applicant: L'Oreal, Paris (FR)

(72) Inventor: Jianxin FENG, Clark, NJ (US)

(21) Appl. No.: 17/008,135

(22) Filed: Aug. 31, 2020

(51) Int. Cl. A61K 8/44; A61K 8/73; A61K 8/362; A61K 8/365; A61K 8/41; A61K 8/46; A61Q 5/02

(52) U.S. Cl. CPC A61K 8/44; A61K 8/731; A61K 8/732; A61K 8/362; A61K 8/365; A61K 2800/596; A61K 8/466; A61Q 5/02; A61K 2800/48; A61K 2800/34; A61K 8/41

(57) ABSTRACT

The disclosure relates to compositions for cleansing keratin materials. The compositions comprise (a) at least one first amphoteric surfactant chosen from alkyl amphoacetates, alkyl amphodiacetates, salts thereof, or mixtures thereof; (b) at least one second amphoteric surfactant chosen from betaines or salts thereof; (c) at least one nonionic surfactant; (d) at least one non-sulfate anionic surfactant; (e) at least one fatty amine; and (f) at least one polysaccharide thickening agent; wherein the composition is free or essentially free of sulfate-based surfactants and optionally free or essentially free of silicones. The disclosure also relates to methods of using the compositions.

### TECHNICAL FIELD

The present disclosure relates to compositions for cleansing keratin materials, and methods of using the compositions.

### BACKGROUND

Conventional personal care cleansing compositions such as shampoo, body wash, facial cleanser, hand soap, etc., typically use sulfate-based surfactants such as sodium lauryl sulfate (SLS) or sodium laureth ether sulfate (SLES). These surfactants are commonly used because they have good foaming and cleansing properties, can be thickened easily, and are relatively inexpensive. However, there have been growing concerns in the marketplace over the negative effects of these or other sulfate-based surfactants, or sulfate-containing surfactants, on the skin and body. For example, sulfate-based surfactants have a tendency to dry out hair and skin, strip dye from color-treated hair, and break down proteins such as keratin, and may cause skin and eye irritation. In addition, SLES may contain dioxanes, byproducts generated in the manufacturing process, which are considered carcinogenic at high enough levels.

In addition, silicones are commonly used in personal care products for their conditioning and cosmetic effects. For example, silicones provide a protective layer on the hair which allows the hair to be easily detangled and combed, and providing smoothness and glossiness. However, silicones can build up on hair layer-by-layer, which can weigh down the hair and make the hair greasy. Furthermore, silicones are not easily degraded, and accordingly their use in personal care products raises environmental concerns.

Furthermore, consumers desire natural compositions for personal care products such as compositions for cleansing hair and skin. There is an increased demand for sustainable, safe, and environmentally friendly “green” compositions that are free of or essentially free of silicones, as well as other synthetic chemical materials for cleansing and/or caring cleansing keratin materials, including hair and skin, and yet provide desirable overall good performance and high safety. However, such “green” compositions can be expensive to produce as their materials must be sourced from natural sources such as plants, as opposed to being high-volume, industrially-produced chemicals. Moreover, it is often difficult to achieve an acceptable balance of desirable cleansing composition performance properties when using naturally-sourced products. For example, the addition of a particular component to a cleansing composition will often enhance one desired property to the detriment of another desired property.

Therefore, cleansing compositions that are free of sulfate-based surfactants and/or silicones are becoming increasingly desirable to consumers. However, there are challenges in developing suitable formulations of cleansing products without the use of sulfate-based surfactants such as sulfate-based anionic surfactants, and/or silicones (“sulfate-free” and/or “silicone-free”). For example, most existing sulfate-free hair cleansing products foam poorly, are opaque, and are not easily thickened. Traditional methods of increasing viscosity of these formulations, such as incorporation of a salt, are not effective with sulfate-free surfactants.

Thus, there is a need to overcome the above described challenges for developing compositions for cleansing keratin materials that meet the consumers' increasing demands for natural, sulfate-free, and silicone-free cleansing products, as well as deliver satisfactory cosmetic properties to the keratin materials.

It has now surprisingly been found that, by using a synergistic combination of components, a composition for cleansing keratin materials that is free or essentially free of sulfate-based surfactants and/or silicone compounds can be prepared, which demonstrates excellent foaming and cleansing properties, as well as good cosmetic properties, and is environmentally friendly.

### BRIEF DESCRIPTION OF FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a graph illustrating a foaming performance of Inventive Composition 1A according to one embodiment of the disclosure in a foaming analysis.

FIG. 2 is a graph illustrating a foaming performance of Inventive Composition 1B according to one embodiment of the disclosure in a foaming analysis.

FIG. 3 is a graph illustrating a foaming performance of Inventive Composition 1C according to one embodiment of the disclosure in a foaming analysis.

FIG. 4 is a graph illustrating a foaming performance of a commercially available shampoo during a foaming analysis.

FIG. 5 is a graph illustrating the foaming performance of Inventive Compositions 1A-1C of FIGS. 1-3 in comparison with the foaming performance of the commercially available shampoo of FIG. 4 in a foaming analysis.

FIG. 6 is a graph illustrating the foam structure analysis of foams generated by Inventive Composition 1A according to one embodiment of the disclosure.

FIG. 7 is a graph illustrating the foam structure analysis of foams generated by Inventive Composition 1B according to one embodiment of the disclosure.

FIG. 8 is a graph illustrating the foam structure analysis of foams generated by Inventive Composition 1C according to one embodiment of the disclosure.

FIG. 9 is a graph illustrating the foam structure analysis of foams generated by a commercially available shampoo.

It is to be understood that the foregoing and following descriptions are exemplary and explanatory only, and are not intended to be restrictive of any subject matter claimed.

### SUMMARY

The present disclosure relates to compositions for cleansing keratin materials and methods of using the compositions. The compositions may be free or essentially free of sulfate-based surfactants and/or silicones.

In exemplary and non-limiting embodiments, the disclosure relates to a composition for cleansing keratin materials, the composition comprising (a) at least one first amphoteric surfactant chosen from alkyl amphoacetates, alkyl amphodiacetates, salts thereof, or mixtures thereof; (b) at least one second amphoteric surfactant chosen from betaines or salts thereof; (c) at least one nonionic surfactant; (d) at least one non-sulfate anionic surfactant; (e) at least one fatty amine; and (f) at least one polysaccharide thickener. The composition is free or essentially free of sulfate-based surfactants, and optionally free or essentially free of silicones and/or cationic polymers. In various embodiments, the at least one first amphoteric surfactant comprises at least one (C8-C20)alkylamphodiacetate, and the at least one second amphoteric surfactant comprises at least one compound chosen from alkyl betaines, amido betaines, or mixtures thereof. In further embodiments, the at least one nonionic surfactant is chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, alkyl and polyalkyl esters of sucrose, optionally polyoxyethylenated alkyl and polyalkyl esters of glycerol, and optionally polyoxyethylenated alkyl and polyalkyl ethers of glycerol. In still further embodiments, the at least one nonionic surfactant is chosen from alkypolyglucosides. In further embodiments, the at least one non-sulfate anionic surfactant is chosen from alkyl sulfonates, alkyl sulfosuccinates, alkyl sulfoacetates, acyl isethionates, alkoxylated monoacids, acyl amino acids, for example acyl taurates, acyl glycinates, acyl glutamates, acyl sarcosinates, salts thereof, or mixtures thereof. In yet further embodiments, the at least one polysaccharide thickener is chosen from gums, celluloses, and starches. The compositions may have a gel-like texture and may be essentially or partially transparent. All components of the composition may be derived from sustainable and environment friendly raw materials, with up to 100% biodegradability.

In various embodiments, compositions according to the disclosure comprise (a) about 0.1% to about 15% of at least one first amphoteric surfactant chosen from alkyl amphoacetates, alkyl amphodiacetates, salts thereof, or mixtures thereof; (b) about 0.1% to about 15% of at least one second amphoteric surfactant chosen from betaines or salts thereof; (c) about 0.1% to about 10% of at least one nonionic surfactant; (d) about 0.01% to about 6% of at least one non-sulfate anionic surfactant; (e) about 0.01% to about 10% of at least one fatty amine; and (f) about 0.01% to about 5% of at least one polysaccharide thickener, all amounts by weight of active material, relative to the composition. In some embodiments, the total amount of amphoteric surfactants ranges from about 5% to about 20% by weight, relative to the total weight of the composition. In still further embodiments, the (a) at least one first amphoteric surfactant chosen from alkyl amphoacetates, alkyl amphodiacetates, salts thereof, or mixtures thereof and the (b) at least one second amphoteric surfactant chosen from betaines or salts thereof are present in the composition in a weight ratio ranging from 1:10 to 10:1, such as about 1:1.

In other exemplary and non-limiting embodiments, the disclosure relates to a composition for cleansing and/or conditioning keratin materials, the composition comprising (a) at least one first amphoteric surfactant chosen (C8-C20)alkyl-amphodiacetates or salts thereof; (b) at least one second amphoteric surfactant chosen from alkyl betaines or salts thereof; (c) at least one nonionic surfactant chosen from alkylpolyglucosides; (d) at least one non-sulfate anionic surfactant chosen from alkyl sulfonates, alkyl sulfosuccinates, acyl taurates, salts thereof, or mixtures thereof; (e) at least one fatty amine; and (f) at least one polysaccharide thickening agent chosen from gums; wherein the composition is free or essentially free of sulfate-based surfactants and silicones, and optionally free or essentially free of cationic polymers. The compositions may have a gel-like texture and may be essentially or partially transparent. All components of the composition may be derived from sustainable and environment friendly raw materials, with up to 100% biodegradability.

In another exemplary and non-limiting embodiment, the disclosure relates to a composition for cleansing and/or conditioning keratin materials, the composition comprising (a) at least one first amphoteric surfactant comprising disodium cocoamphodiacetate; (b) at least one second amphoteric surfactant comprising cocamidopropyl betaine/coco-betaine; (c) at least one nonionic surfactant comprising caprylyl/capryl glucoside; (d) at least one non-sulfate anionic surfactant chosen from sodium C14-16 olefin sulfonates; (e) stearamidopropyl dimethylamine; and (f) at least one polysaccharide thickening agent chosen from gums; wherein the composition is free or essentially free of sulfate-based surfactants and silicones, and optionally free or essentially free of cationic polymers.

In yet a further exemplary and non-limiting embodiment, the disclosure relates to a method of cleansing and/or conditioning a keratin material, the method comprising (i) applying to the keratin material a composition comprising (a) at least one first amphoteric surfactant chosen from alkyl amphoacetates, alkyl amphodiacetates, salts thereof, or mixtures thereof; (b) at least one second amphoteric surfactant chosen from betaines or salts thereof; (c) at least one nonionic surfactant; (d) at least one non-sulfate anionic surfactant; (e) at least one fatty amine; and (f) at least one polysaccharide thickening agent; wherein the composition is free or essentially free of sulfate-based surfactants, and optionally free or essentially free of silicones and/or cationic polymers; and (ii) subsequently rinsing the composition from the keratin material.

### DESCRIPTION

The disclosure relates to compositions for cleansing and/or conditioning keratin materials, and methods of using the compositions. The compositions may be free or essentially free of sulfate-based surfactants and/or silicones.

I. Compositions

In exemplary and non-limiting embodiments, compositions according to the disclosure comprise (a) at least one first amphoteric surfactant chosen from alkyl amphoacetates, alkyl amphodiacetates, salts thereof, or mixtures thereof; (b) at least one second amphoteric surfactant chosen from betaines or salts thereof; (c) at least one nonionic surfactant; (d) at least one non-sulfate anionic surfactant; (e) at least one fatty amine; and (f) at least one polysaccharide thickener.

Compositions according to the disclosure are free or essentially free of sulfate-based surfactants. In some embodiments, the compositions are free or essentially free of silicones. In some embodiments, the compositions are free or essentially free of cationic surfactants.

Amphoteric Surfactants

Compositions according to the disclosure comprise at least one first amphoteric surfactant chosen from alkyl amphoacetates, alkyl amphodiacetates, or salts thereof, and at least one second amphoteric surfactant chosen from betaines or salts thereof.

Alkyl Amphoacetates and Alkyl Amphodiacetates

By way of example only, useful alkyl amphoacetates and alkyl amphodiacetates include those of Formula (Ia) or (Ib):

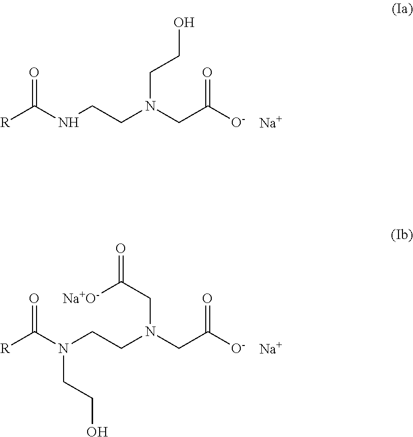

wherein R is an alkyl group having 8-18 carbon atoms.

Although sodium is shown as the cation in the above formulae, the cation may be any alkali metal ion, such as sodium or potassium, an ammonium ion, or an alkanolammonium ion such as monoethanolammonium or triethanolammonium ions. A non-limiting example is sodium lauroamphoacetate.

Additional non-limiting examples of alkyl amphoacetates and alkyl amphodiacetates include those of formula (Ic):

Ra′-CON(Z)CH2-(CH2)m′-N(B)(B′) (Ic)
wherein:
B represents —CH2CH2OX′, with X′ representing —CH2-COOH, CH2-COOZ′, —CH2CH2-COOH, —CH2CH2-COOZ′, or a hydrogen atom;
B′ represents —CH2)z-Y′, with z=1 or 2, and Y′ representing —COOH, —COOZ′, —CH2-CHOH—SO3H or —CH2-CHOH—SO3Z′;
m′ is equal to 0, 1 or 2;
Z represents a hydrogen atom or a hydroxyethyl or carboxymethyl group;
Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanol-amine, diisopropanolamine or triisopropanolamine, 2-amino-2-methyl-1-propanol, 2-amino-2-methyl-1,3-propanediol and tris(hydroxy-methyl)aminomethane; and
Ra′ represents a (C10-C30)alkyl or alkenyl group of an acid Ra′COOH preferably present in hydrolyzed linseed oil or coconut oil, an alkyl group, in particular a C17 alkyl group, and its iso form, or an unsaturated C17 group.

Exemplary compounds of formula (Ic) include (C8-C20)alkylamphoacetates and (C8-C20)alkylamphodiacetates, such as disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylampho-dipropionate, disodium caprylomphodipropionate, lauroamphodipropionic acid, or cocoamphodipropionic acid. For example, disodium cocoamphodiacetate supplied by Rhodia under the name MIRANOLI C₂M can be used.

The total amount of alkyl amphoacetates and/or alkyl amphodiacetates, or salts thereof, in the composition may vary, but is typically from about 0.1% to about 15% by weight, including all subranges therebetween, such as from about 0.1% to about 10%, from about 0.1% to about 8%, from about 0.1% to about 6%, from about 1% to about 15%, from about 1% to about 10%, from about 1% to about 8%, or from about 1% to about 6%, by weight, relative to the total weight of the composition. For example, the total amount of alkyl amphoacetates and/or alkyl amphodiacetates, or salts thereof, may range from about 1% to about 10%, from about 1% to about 9%, from about 1% to about 8%, from about 1% to about 7%, from about 1% to about 6%, from about 1% to about 5%, from about 1% to about 4%, from about 2% to about 10%, from about 2% to about 9%, from about 2% to about 8%, from about 2% to about 7%, from about 2% to about 6%, from about 2% to about 5%, from about 2% to about 4%, from about 3% to about 10%, from about 3% to about 9%, from about 3% to about 8%, from about 3% to about 7%, from about 3% to about 6%, from about 3% to about 5%, or from about 3% to about 4%, relative to the total weight of the composition.

### Betaines

In various embodiments, non-limiting examples of betaines or salts thereof that the at least one second amphoteric surfactant may comprise alkyl betaines, amido betaines, or mixtures thereof. Possible betaines may be derived from a variety of natural oils or fatty acids.

In some embodiments, exemplary useful betaines include, but are not limited to, those of the following formulae (IIa-IId):

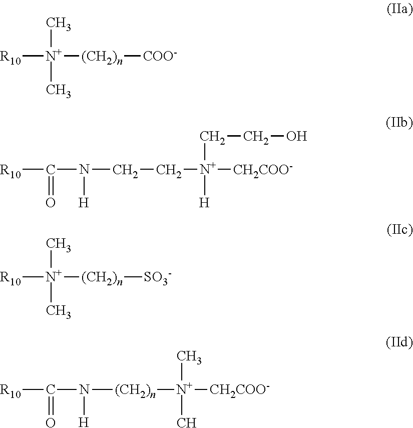

wherein:
R₁₀ is an alkyl group having from 8-18 carbon atoms; and
n is an integer from 1 to 3.

Particularly useful betaines include, for example, coco-betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, or mixtures thereof. Typically, at least one betaine compound is selected from coco betaine, cocamidopropyl betaine, behenyl betaine, capryl/capramidopropyl betaine, and lauryl betaine, and mixtures thereof. In one embodiment, preferred betaines include coco-betaine and cocamidopropyl betaine.

The total amount of betaines or salts thereof in the composition may vary but is typically from about 0.1% to about 15% by weight, including all subranges therebetween, such as from about 0.1% to about 10%, from about 0.1% to about 8%, from about 0.1% to about 6%, from about 1% to about 15%, from about 1% to about 10%, from about 1% to about 8%, or from about 1% to about 6%. For example, the total amount of betaines or salts thereof may range from about 1% to about 10%, from about 1% to about 9%, from about 1% to about 8%, from about 1% to about 7%, from about 1% to about 6%, from about 1% to about 5%, from about 1% to about 4%, from about 2% to about 10%, from about 2% to about 9%, from about 2% to about 8%, from about 2% to about 7%, from about 2% to about 6%, from about 2% to about 5%, from about 2% to about 4%, from about 3% to about 10%, from about 3% to about 9%, from about 3% to about 8%, from about 3% to about 7%, from about 3% to about 6%, from about 3% to about 5%, or from about 3% to about 4%, relative to the total weight of the composition.

In some embodiments, the amphoteric surfactants are the predominant type of surfactant in the surfactant system, i.e., there is a higher percentage of amphoteric surfactants than any other single type of surfactant in the composition. Moreover, in some instances, the total amount of amphoteric surfactants in the surfactant system is higher than the total amount of all other surfactant types in the surfactant system. In other words, the phrase “all other surfactants” means any and all surfactants in the composition other than amphoteric surfactants.

In various exemplary embodiments, the total amount of the amphoteric surfactants in compositions according to the disclosure may vary, but typically ranges from about 0.1% to about 30%, including all subranges therebetween, such as from about 0.1% to about 25%, from about 0.1% to about 20%, from about 0.1% to about 15%, from about 0.1% to about 10%, from about 1% to about 30%, from about 1% to about 25%, from about 1% to about 20%, from about 1% to about 15%, from about 1% to about 10%, from about 5% to about 30%, from about 5% to about 25%, from about 5% to about 20%, from about 5% to about 15%, from about 5% to about 10%, from about 8% to about 30%, from about 8% to about 25%, from about 8% to about 20%, from about 8% to about 15%, or from about 8% to about 10% by weight, relative to the total weight of the composition.

The ratio of the amount of the at least one first amphoteric surfactant to the amount of the at least one second amphoteric surfactant may range from about 1:10 to about 10:1. For example, the ratio of the amounts of the at least one first amphoteric surfactant to the at least one second amphoteric surfactant, or the at least one second amphoteric surfactant to the at least one first amphoteric surfactant, may be about 1:10, about 1:9, about 1:8, about 1:7, about 1:6, about 1:5, about 1:4, about 1:3, about 1:2, or about 1:1. In a preferred embodiment, the total amount of first amphoteric surfactants and the total amount of the second amphoteric surfactants are approximately the same, i.e. about 1:1.

In various exemplary embodiments, the total amount of amphoteric surfactants is greater than the combined amount of non-ionic and non-sulfate anionic surfactants.

### Nonionic Surfactants

Compositions according to the disclosure comprise at least one nonionic surfactant. The at least one nonionic surfactant may optionally be derived from plants. In certain embodiments, the nonionic surfactants may be chosen especially from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, for example alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof.

Exemplary and nonlimiting alkyl and polyalkyl esters of poly(ethylene oxide) include those containing at least one C8-C30 alkyl radical, with a number of ethylene oxide (EO) units ranging from 2 to 200. Mention may be made, for example, of PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.

Exemplary and nonlimiting alkyl and polyalkyl ethers of poly(ethylene oxide) include those containing at least one C8-C30 alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.

Exemplary and nonlimiting polyoxyethylenated alkyl and polyalkyl esters of sorbitan include those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

Exemplary and nonlimiting polyoxyethylenated alkyl and polyalkyl ethers of sorbitan include those with a number of ethylene oxide (EO) units ranging from 0 to 100.

Exemplary and nonlimiting alkyl and polyalkyl glucosides or polyglucosides include those containing an alkyl group comprising from 6 to 30 carbon atoms and preferably from 6 to 18 or even from 8 to 16 carbon atoms, and containing a glucoside group preferably comprising from 1 to 5 and especially 1, 2, or 3 glucoside units.

In certain embodiments, exemplary and useful alkyl polyglucosides include those having the following formula (III):

R¹—O—(R²O)ₙ—Z₍ₓ₎ (III)

wherein:
R¹ is an alkyl group having 8-18 carbon atoms;
R² is an ethylene or propylene group;
Z is a saccharide group with 5-6 carbon atoms;
n is an integer ranging from 0 to 10; and
x is an integer ranging from 1 to 5

The alkylpolyglucosides may be chosen, for example, from decylglucoside, for instance the product sold under the name MYDOL10® by the company Kao Chemicals or the product sold under the name PLANTACARE® 2000 UP by the company BASF and the product sold under the name ORAMIX™ NS 10 by the company SEPPIC; caprylyl/capryl glucoside, for instance the product sold under the name PLANTACARE® KE 3711 by the company Cognis or ORAMIX™ CG 110 by the company SEPPIC; laurylglucoside, for instance the product sold under the name PLANTACARE® 1200 UP by the company BASF or PLANTAREN 1200 N® by the company BASF; cocoglucoside, for instance the product sold under the name PLANTACARE® 818 UP by the company BASF; caprylylglucoside, for instance the product sold under the name PLANTACARE® 810 UP by the company BASF, octyl glucoside, and mixtures thereof.

Exemplary and nonlimiting alkyl and polyalkyl esters of sucrose that may be mentioned are Crodesta™ F150, sucrose monolaurate sold under the name Crodesta SL 40, and the products sold by Ryoto Sugar Ester, for instance sucrose palmitate sold under the reference Ryoto™ Sugar Ester P1670, Ryoto™ Sugar Ester LWA 1695 or Ryoto Sugar™ Ester 01570. Sucrose monooleate, monomyristate and monostearate are also suitable for use.

Exemplary and nonlimiting (poly)oxyethylenated alkyl and polyalkyl esters of glycerol include those with a number of ethylene oxide (EO) units ranging from 0 to 100 and a number of glycerol units ranging from 1 to 30. Mention may be made, for example, of hexaglyceryl monolaurate, PEG-30 glyceryl stearate, polyglyceryl-2 laurate, polyglyceryl-10 laurate, polyglyceryl-10 stearate, polyglyceryl-10 oleate, PEG-7 glyceryl cocoate and PEG-20 glyceryl isostearate.

Exemplary and nonlimiting (poly)oxyethylenated alkyl and polyalkyl ethers of glycerol include those with a number of ethylene oxide (EO) units ranging from 0 to 100 and a number of glycerol units ranging from 1 to 30. Examples that may be mentioned include Nikkol Batyl Alcohol 100 and Nikkol Chimyl Alcohol 100.

In various exemplary embodiments, the total amount of the one or more nonionic surfactants may be about 0.1% to about 10%, based on the total weight of the composition, including all ranges and subranges therebetween. For example, the total amount of the one or more nonionic surfactants may range from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 1% to about 10%, from about 1% to about 9%, from about 1% to about 8%, from about 1% to about 7%, from about 1% to about 6%, from about 1% to about 5%, from about 1% to about 4%, or from about 1% to about 3% by weight, relative to the total weight of the hair treatment composition. In some embodiments, the at least one nonionic surfactant is present in an amount ranging from about 1.5% to about 5% by weight, based on the total weight of the composition.

### Non-Sulfate Anionic Surfactants

Compositions according to the disclosure typically comprise at least one non-sulfate anionic surfactant. In one embodiment, the composition may be free or essentially free of any anionic surfactant.

Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof. Non-limiting examples of these non-sulfate anionic surfactants are provided below.

### Acyl Isethionates

Non-limiting examples of useful acyl isethionates and their salts include those of formula (IVa) and (IVb):

RCOOCHR¹CHR²X⁻M⁺ (IVa)

RCOOCHR¹CHR²X⁻Na⁺ (IVb)

wherein:
R, R′, and R² are each independently chosen from H or an alkyl chain having 1-24 carbon atoms, said chain being saturated or unsaturated, linear or branched;
X is COO⁻ or SO₃⁻; and
M is any suitable cation.

Although the cation may be chosen from any suitable cation including, for example, alkali metal ion such as sodium or potassium, ammonium ions, or alkanolammonium ions such as monoethanolammonium or triethanolammonium ions, sodium is a preferred cation. In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

### Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

### Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, sulfonates of alkylphenolpolyglycol ethers, alkylbenzenesulfonates, phenylalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful:

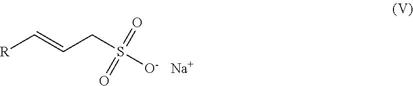

wherein R is selected from H or alkyl chain that has 1-24 carbon atoms, preferably 6-24 carbon atoms, more preferably, 8 to 20 carbon atoms, said chain being saturated or unsaturated, linear or branched, substituted or unsubstituted. Sodium is shown as the cation in the above formula (V) but the cation may be may be chosen from any suitable cation including, for example, alkali metal ion such as sodium or potassium, ammonium ions, or alkanolammonium ions such as monoethanolammonium or triethanolammonium ions.

In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, or mixtures thereof. In certain embodiments, C₁₀-C₂₄ olefin sulfonates and salts thereof may be preferred. A non-limiting example of a C₁₀-C₂₄ olefin sulfonate that can be used is sodium C₁₄₋₁₆ olefin sulfonate.

### Alkyl Sulfosuccinates

Non-limiting examples of useful alkyl sulfosuccinates and their salts include those of formula (VI):

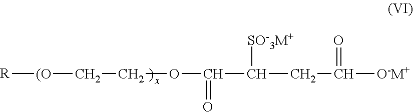

wherein:
R is a straight or branched chain alkyl or alkenyl group having 10 to 22 carbon atoms, preferably 10 to 20 carbon atoms;
x is a number that represents the average degree of ethoxylation, and can range from 0 to about 5, preferably from 0 to about 4, and most preferably from about 2 to about 3.5; and
M, which can be the same or different, is chosen from any suitable monovalent cation.

In some embodiments, cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

Alkyl Sulfoacetates

Non-limiting examples of alkyl sulfoacetates and their salts include, for example, alkyl sulfoacetates such as C4-C18 fatty alcohol sulfoacetates and/or salts thereof. In some embodiments, a sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

### Alkoxylated Monoacids

Non-limiting examples of alkoxylated monoacids include compounds corresponding to formula (VII):

R—O[CH₂O]ᵤ[(CH₂)ₓCH(R′)(CH₂)_{y}(CH₂)_{z}O]ᵥ[CH₂CH₂O]_{w}CH₂COOH (VII)

wherein:
R is a hydrocarbon radical containing from about 6 to about 40 carbon atoms;
R′ represents hydrogen or alkyl;
u, v, and w, which may be identical or different, independently represent numbers from 0 to 60;
x, y, and z, which may be identical or different, independently represent numbers from 0 to 13; and
the sum of x+y+z>0.

Compounds corresponding to formula (VII) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. For example, R may be a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, may be chosen from a number ranging from 2 to 20, such as a number ranging from 3 to 17, or a number ranging from 5 to 15. Further still, x, y, z, independently of one another, may be chosen from a number ranging from 0 to 13, such as a number ranging from 1 to 10, or a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof. In some cases, preferred ethoxylated acids include Oleth-10 Carboxylic Acid, Laureth-5 Carboxylic Acid, Laureth-11 Carboxylic Acid, or mixtures thereof.

### Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt.

Non-limiting examples of useful acyl amino acids include those of formula (VIII):

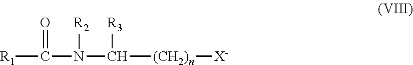

wherein:
R¹, R², and R³ are each independently selected from H or an alkyl chain having 1-24 carbon atoms, said chain being saturated or unsaturated, linear or branched, substituted or unsubstituted;
n ranges from 0 to 30; and
X is COO⁻ or SO₃⁻.

### Acyl Taurates

Non-limiting examples of acyl taurates include those of formula (IX):

RCO—NR¹CHR²CHR³SO₃Na (IX)

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO— represents the coconut acid moiety. Non-limiting examples of acyl taurate salts include sodium cocoyl taurate and sodium methyl cocoyl taurate.

### Acyl Glycinates

Non-limiting examples of useful acyl glycinates include those of formula (X):

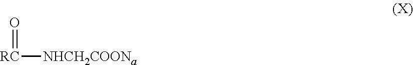

wherein R is an alkyl chain of 8 to 16 carbon atoms. Sodium is shown as the cation in the above formula (X), but the cation may be any alkali metal ion such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate.

### Acyl Glutamates

Non-limiting examples of useful acyl glutamates include those of formula (XI):

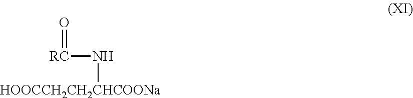

wherein R is an alkyl chain of 8 to 16 carbon atoms. Sodium is shown as the cation in the above formula (XI) but the cation may be any alkali metal ion such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

In various embodiments, the total amount of non-sulfate anionic surfactants in the compositions may vary, but is typically from about 0.01% to about 6% by weight, including all subranges therebetween, such as from about 0.01% to about 4%, from about 0.01% to about 3%, from about 0.01% to about 2%, from about 0.01% to about 1%, from about 0.5% to about 5%, from about 0.5% to about 4%, from about 0.5% to about 3%, from about 0.5% to about 2%, from about 1% to about 5%, from about 1% to about 4% by weight, relative to the total weight of the composition. In some particular embodiments, the total amount of non-sulfate anionic surfactants in the compositions may ranges from about 0.05% to about 4% by weight, relative to the total weight of the composition.

### Fatty Amines

Compositions according to the disclosure comprise at least one fatty amine. The useful “fatty amines” include primary, secondary, or tertiary fatty amines, which are optionally (poly)oxyalkylenated, or salts thereof.

In some embodiments, the fatty amines according to the present disclosure may comprise at least one C₆-C₃₀ hydrocarbon-based chain. In some embodiments, the fatty amines according to the disclosure are not quaternized. In some embodiments, the fatty amines according to the disclosure are not (poly)oxyalkylenated.

In some exemplary embodiments, the composition according to the disclosure comprises at least one fatty amine chosen from tertiary fatty amines. The composition according to the disclosure may comprise one or more tertiary fatty amines chosen from fatty amidoamines.

The fatty amines that may be used in the context of the present disclosure may be chosen from the fatty amines having the formula (XII) below:

RN(R′)₂ (XII)

in which R represents a monovalent hydrocarbon-based radical containing from 6 to 30 carbon atoms, preferably from 8 to 24 carbon atoms, and in particular a linear or branched, saturated or unsaturated and substituted or unsubstituted C₆-C₃₀ and preferably a C₈-C₂₄ alkyl radical, preferably a linear or branched C₆-C₃₀ and better still C₈-C₂₄ alkyl radical, or a linear or branched C₆-C₃₀ and preferably C₈-C₂₄ alkenyl radical; and R′, which may be identical or different, represent a linear or branched, saturated or unsaturated and substituted or unsubstituted monovalent hydrocarbon-based radical containing less than 6 carbon atoms, preferably from 1 to 4 carbon atoms, preferably a methyl radical.

The fatty amines corresponding to formula (XII) may be chosen, for example, from dimethyllauramine, dimethylbehenamine, dimethylcocamine, dimethylmyristamine, dimethylpalmitamine, dimethylstearamine, dimethyltallowamine, dimethylsoyamine, and mixtures thereof.

The fatty amines that may be used in the context of the disclosure may also be chosen from fatty amidoamines, such as the fatty amidoamines having the formula (XIII) below:

RCONHR″N(R′)₂ (XIII)

in which R represents a monovalent hydrocarbon-based radical containing from 5 to 29 carbon atoms, preferably from 7 to 23 carbon atoms, and in particular a linear or branched, saturated or unsaturated and substituted or unsubstituted C₅-C₂₉ and preferably a C₇-C₂₃ alkyl radical, preferably a linear or branched C₅-C₂₉ and better still C₅-C₂₃ alkyl radical, or a linear or branched C₅-C₂₉ and preferably C₇-C₂₃ alkenyl radical; R″, which may be identical or different, represent a divalent hydrocarbon-based radical containing less than 6 carbon atoms, preferably 2 or 3 carbon atoms; and R′, which may be identical or different, represent a linear or branched, saturated or unsaturated and substituted or unsubstituted monovalent hydrocarbon-based radical containing less than 6 carbon atoms, preferably from 1 to 4 carbon atoms, preferably a methyl radical.

The fatty amines corresponding to formula (XIII) are chosen, for example, from oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, avocadoamidopropyl dimethylamine, cocamidopropyl dimethylamine, minkamidopropyl dimethylamine, oatamidopropyl dimethylamine, sesamidopropyl dimethylamine, tallamidopropyl dimethylamine, olivamidopropyl dimethylamine, palmitamidopropyl dimethylamine, stearamidoethyldiethylamine, brassicamidopropyl dimethylamine, and mixtures thereof.

In a preferred embodiment, the fatty amine is stearamidopropyl dimethylamine, such as that sold by the company Inolex Chemical Company under the name Lexamine S13.

In various embodiments, the total amount of fatty amines in the compositions may vary, but is typically from about 0.01% to about 10% by weight, including all subranges therebetween, such as from about 0.01% to about 5%, from about 0.01% to about 4%, from about 0.01% to about 3.5%, from about 0.01% to about 3%, from about 0.01% to about 2.5%, from about 0.01% to about 2%, from about 0.1% to about 5%, from about 0.1% to about 4%, from about 0.1% to about 3.5%, from about 0.1% to about 3%, from about 0.1% to about 2.5%, from about 0.1% to about 2%, from about 1% to about 5%, from about 1% to about 4%, from about 1% to about 3.5%, from about 1% to about 3%, from about 1% to about 2.5%, or from about 1% to about 2%, by weight, relative to the total weight of the composition. In some particular embodiments, the total amount of fatty amines in the compositions may ranges from about 0.5% to about 4% by weight, relative to the total weight of the composition.

### Polysaccharide Thickeners

The compositions further comprise at least one polysaccharide thickener (also referred to as thickening agents or viscosity modifying agents). Polysaccharide thickeners are polymers which exhibit monosaccharides or disaccharides as base units. The polysaccharide thickeners which can be used in the compositions according to the present invention include, by way of example only, gums, celluloses, and starches.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxyethylcelluloses, hydroxypropylmethylcellulose, or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose.

Non-limiting examples of starches include modified starches, starch-based polymers, methylhydroxypropyl starch, potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In various exemplary embodiments, the total amount of the one or more polysaccharide thickener may vary, but is typically ranges from about 0.01% to about 5%, including all subranges therebetween, such as from about 0.01% to about 4%, from about 0.01% to about 3%, from about 0.01% to about 2%, from about 0.01% to about 1.5%, from about 0.01% to about 1%, from about 0.01% to about 0.5%, from about 0.1% to about 5%, from about 0.1% to about 4%, from about 0.1% to about 3%, from about 0.1% to about 2%, from about 0.1% to about 1.5%, from about 0.1% to about 1%, or from about 0.1% to about 0.5%, by weight, relative to the total weight of the composition. In at least certain embodiments, the composition comprises a polysaccharide thickener in an amount less than about 1%.

### Solvents

Compositions according to the disclosure comprise a solvent. The solvent may be chosen from water, non-aqueous solvents, or mixtures thereof.

In some embodiments, the solvent comprises, consists essentially of, or consists of water. The total amount of water in the compositions may vary depending on the type of composition and the desired consistency, viscosity, etc.

In certain embodiments, the composition comprises one or more non-aqueous solvents, for example, glycerin, C₁₋₄ alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

The solvent may be present in the composition in an amount ranging from about 60% to about 98% by weight, relative to the total weight of the composition, including all ranges and subranges therebetween. For example, in one embodiment, the total amount of solvent may be about 70% to about 95%, about 70% to about 90%, about 70% to 85%, or about 70% to 80% by weight, relative to the total weight of the composition. In certain embodiments, the solvent is primarily comprised of water, such as from about 90% to about 99%, or about 95% to about 99%, of the total solvent.

### Conditioning Agents

Compositions according to the disclosure may optionally comprise at least one conditioning agent other than the at least one fatty amine. However, in some embodiments, the compositions are free or essentially free of conditioning agents other than the at least one fatty amine.

In various exemplary embodiments, the at least one conditioning agent may be chosen from non-silicone fatty compounds. The term “non-silicone fatty compound” means a fatty compound that does not contain any silicon atoms (Si). Non-limiting examples of non-silicone fatty compounds include oils, mineral oil, fatty alcohols, fatty acids, fatty alcohol derivatives, fatty acid derivatives (such as alkoxylated fatty acids or polyethylene glycol esters of fatty acids, propylene glycol esters of fatty acids, butylene glycol esters of fatty acids, esters of neopentyl glycol and fatty acids, polyglycerol/glycerol esters of fatty acids, glycol diesters or diesters of ethylene glycol, fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of the fatty alcohols, fatty acids, fatty alcohol derivatives, and fatty acid derivatives are found in International Cosmetic Ingredient Dictionary, Sixteenth Edition, 2016, which is incorporated by reference herein in its entirety.

In some instances, the non-silicone fatty compounds include one or more waxes. The waxes generally have a melting point of from 35-120° C., at atmospheric pressure. Non-limiting examples of waxes in this category include for example, synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, sunflower seed wax 20 (Helianthus annuus), acacia decurrents flower wax, or a mixture thereof.

The waxes capable of being used as non-silicone fatty compounds may be animal waxes, such as beeswax; vegetable waxes, such as sunflower seed (helianthus annuus), carnauba, candelilla, ouricury or japan 25 wax or cork fiber or sugarcane waxes; mineral waxes, for example paraffin or lignite wax or microcrystalline waxes or ozokerites; synthetic waxes, including polyethylene waxes, and waxes obtained by the Fischer-Tropsch synthesis.

In some instance, the non-silicone fatty compounds include one or more non-silicone oils. The term “oil” as used herein describes any material which is substantially insoluble in water. Suitable non-silicone oils include, but are not limited to, natural oils, such as coconut oil; hydrocarbons, such as mineral oil and hydrogenated polyisobutene; fatty alcohols, such as octyldodecanol; esters, such as C12-C15 alkyl benzoate; diesters, such as propylene dipelarganate; and triesters, such as glyceryl trioctanoate. Suitable low viscosity oils have a viscosity of 5-100 mPas at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity oils generally have a viscosity of 200-1,000,000, or 100,000-250,000, mPas at 25° C. Such oils include castor oil, lanolin and 15 lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

In various exemplary embodiments, the conditioning agents may be chosen from cationic polymers, although in certain embodiments the compositions are free or essentially free of cationic polymers. The term “cationic polymer” means any polymer comprising at least one cationic group and/or at least one group that may be ionized into a cationic group.

Cationic polymers useful in the compositions may include, homopolymers and copolymers derived from acrylic or methacrylic esters or amides, copolymers of cellulose or cellulose derivatives grafted with a water-soluble quaternary ammonium monomer, polymers of piperazinyl units and of divalent alkylene or hydroxyalkylene radicals, cyclopolymers of alkyldiallylamine and of dialkyldiallylammonium, quaternary diammonium polymers, polyquaternary ammonium polymers, quaternary polymers of vinylpyrrolidone and of vinylimidazole, vinylamide homopolymers or copolymers, cationic polyurethane derivatives and mixture thereof.

Cationic polymers that may be used in the context of the disclosure include, for example, cationic proteins or cationic protein hydrolysates, polyalkyleneimines, such as polyethyleneimines, polymers containing vinylpyridine or vinylpyridinium units, and chitin derivatives.

In various embodiments, useful cationic polymers according to the present disclosure may include, but are not limited to: polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, or guar hydroxypropyltrimonium chloride, and mixtures thereof.

In an embodiment, the cationic conditioning agent is chosen from Polyquaternium-67, Polyquaternium-10, Polyquaternium-37, Polyquaternium-7, or mixtures thereof. Polyquaternium-37 may be commercially available from BASF under the tradename of SALCARE SC 96 (comprising Polyquaternium-37 (and) Propylene Glycol Dicaprylate/Dicaprate (and) PPG-1 Trideceth-6).

In various embodiments, the total amount of at least one conditioning agent in the compositions may vary, but is typically from about 0.01% to about 10% by weight, including all subranges therebetween, such as from about 0.01% to about 8%, from about 0.01% to about 5%, from about 0.01% to about 3%, from about 0.01% to about 1%, from about 0.5% to about 10%, from about 0.5% to about 8%, from about 0.5% to about 5%, from about 1% to about 10%, from about 1% to about 8%, from about 1% to about 5% by weight, relative to the total weight of the composition. In some embodiments, the total amount of conditioning agents are present in an amount ranging from about 0.05% to about 5% by weight, based on the total weight of the composition.

### Active Agents

In various embodiments, compositions according to the disclosure may optionally comprise at least one active agent such as an acid or sodium hydroxide, or mixture thereof, to provide optimized strengthening benefits to the hair. Non-limiting examples of useful acids include glycolic acid, lactic acid, malic acid, tartaric acid, citric acid, ascorbic acid, mandelic acid, azelaic acid, glyceric acid, tartronic acid, gluconic acid, benzylic acid, pyruvic acid, 2-hydroxybutyric acid, salicylic acid, trichloroacetic acid, or mixtures thereof.

The acids are typically non-polymeric and may have one (mono), two (di), or three (tri) carboxylic acid groups (—COOH). The non-polymeric mono-, di-, and tricarboxylic acids, and/or salts thereof, typically have a molecular weight of less than about 500 g/mol, less than about 400 g/mol, or less than about 300 g/mol.

Non-limiting examples of monocarboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof.

Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof.

Non-limiting examples of tricarboxylic acids include citric acid, isocitric acid, aconitric acid, propane-1,2,3-tricarboxylic acid, benzene-1,3,5-tricarboxylic acid, a salt thereof, and a mixture thereof.

If present, the total amount of the one or more active agents may vary but typically ranges from about 0.0001% to about 10%, such as from about 0.0001% to about 5%, about 0.0001% to about 1%, about 0.001% to about 10%, about 0.001% to about 5%, about 0.001% to about 1% by weight, about 0.01% to about 10%, about 0.01% to about 5%, about 0.01% to about 1%, about 0.1% to about 10%, about 0.1% to about 5%, or about 0.1% to about 1% by weight, based on the total weight of the composition. For example, the total amount of the one or more acids may range from about 0.0001% to about 0.5% by weight, based on the total weight of the composition.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), and a mixture thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, and a mixture thereof.

The total amount of the one or more preservatives, when present, may vary. In some cases, the total amount of the one or more preservatives is about 0.01% to about 5%, about 0.01% to about 4%, about 0.15% to about 1%, or about 1% to about 3%, by weight, relative to the total weight of the composition.

### Auxiliary Components

Compositions according to the disclosure may be in any suitable form, for example a dispersion, such as a gel. Thus, compositions according to the disclosure may optionally comprise any auxiliary component suitable for use in such compositions. Such components may include, but are not limited to, dyes/pigments, film forming agents or polymers, humectants and moisturizing agents, fatty substances, thickeners other than polysaccharide thickeners, fillers, structuring agents, shine agents, antioxidants or reducing agents, penetrants, sequestrants, fragrances, buffers, dispersants, plant extracts, preserving agents, opacifiers, sunscreen agents, vitamins, pH adjusting agents, and antistatic agents.

Optional auxiliary components may be present in an amount ranging up to about 15% by weight, relative to the total weight of the composition.

Optionally, the compositions may comprise up to 100% biodegradable, sustainable, and/or environmentally friendly raw materials.

The compositions may be transparent or semi-transparent, and their viscosities may vary but may be often similar to or greater than that of conventional cleansing, shampooing, and/or conditioning compositions. For example, in some embodiments, the compositions according to the present disclosure may range from thickened liquid to a thick gel-like texture. Accordingly, in some instances, the viscosity of a composition disclosed herein may be less than about 100 seconds, such as less than 80 seconds, or from about 10 seconds to about 50 seconds when measured using Ford cup 8.

The compositions may have a pH less than or equal to 7, such as less than or equal to 6, such as between 4 and 6 or between 5 and 6.

In various embodiments, the compositions according to the present disclosure are stable, meaning that no phase separation or significant change in pH or viscosity is seen when stored at a temperature ranging from about 4° C. to about 45° C. for at least about 8 weeks.

In at least some embodiments, compositions according to the disclosure may be mild, display good foaming properties, good detangling and combing properties, good antistatic properties, and/or good stability. The compositions may impart one or more properties such as smoothness, conditioning, excellent detangling, anti-frizz, ease of shaping and/or combing, anti-static, clean and/or smooth appearance, with no weigh-down.

### II. Methods

The present disclosure also relates to methods for cleansing and/or conditioning keratin materials, especially the skin, hair, and/or the scalp, with the compositions disclosed herein. Without limitation, methods of cleansing and/or conditioning keratin materials according to the disclosure may include applying a sufficient amount, or an effective amount, of a composition disclosed herein to a keratin material, such as skin, hair, or scalp, which may be wet, damp, or dry, optionally allowing the composition to remain on the keratin material for a desired amount of time, and optionally rinsing the composition from the keratin material. The composition may optionally be lathered before application to the keratin materials, e.g. in the hands, or may be lathered while on the keratin materials.

Due to the cleansing and conditioning properties of the compositions, in some instances, the compositions may be designated as a “shampoo,” a “conditioning shampoo,” or an “all-in-one conditioning and shampooing composition.” The compositions may also be a face and/or body wash, or both a hair and face and/or body wash. In certain embodiments, compositions of the instant disclosure are particularly useful for cleansing and conditioning hair. Additionally, the compositions provide a variety of desirable cosmetic and styling benefits to the hair, for example, smoothness without weight-down, detangling, and anti-frizz. As such, the compositions are useful in methods for cleansing hair, methods of conditioning hair, and methods for imparting smoothness, detangling, and/or frizz control to hair. Accordingly, the instant disclosure encompasses methods for treating hair with the compositions of the instant disclosure.

Such methods typically include applying an effective amount of a composition of the instant disclosure to the hair, allowing the composition to remain on the hair for a period of time, and subsequently rinsing the composition from the hair. Usually, the composition is merely allowed to remain on the hair for a period of time sufficient to incorporate the composition throughout the hair, for example, by lathering the composition throughout the hair using one's hands.

The amount of time is sufficient for the composition to interact with the hair and any dirt, oil, contamination, etc., that may exist on the hair so that when rinsed, the dirt, oil, contamination, etc., can be effectively removed from the hair and the conditioning agents of the composition can interact with the hair to condition it. Thus, the composition may be allowed to remain on the hair for about 5 seconds to about 30 minutes, about 5 seconds to about 15 minutes, about 5 seconds to about 10 minutes, about 5 seconds to about 5 minutes, about 10 seconds to about 5 minutes, or about 10 seconds to about 3 minutes.

As is common when using shampoo and/or conditioning compositions, the hair may be wetted or rinsed with water prior to application of a composition disclosed herein. Having water already in the hair may be helpful for creating lather when applying the compositions because the water interacts with the surfactants.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

Throughout the disclosure, if the term “a mixture thereof,” or “a combination thereof” including variants, is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin materials” and “applying a composition onto hair” and variations of these phrases are intended to mean contacting the keratin materials including hair and skin, with at least one of the compositions of the disclosure, in any manner. It may also mean contacting the keratin materials in an effective amount.

Unless otherwise indicated, all percentages herein are by weight, relative to the weight of the total composition.

As used herein, the term “conditioning” means imparting to hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work, and consumer perception.

As used herein, “cosmetic composition” encompasses many types of compositions for application to keratin materials such as skin or hair, for example, hair lotions, hair creams, hair gel creams, hair conditioners, hair masques (masks), etc., which can be used either as leave-on or rinse-off treatments or products.

As used herein, the term “non-sulfate-based” or “non-sulfate” anionic surfactants means that the surfactant does not comprise a sulfate group.

As used herein, the term “organic” means a material that is produced substantially without or essentially without the use of synthetic materials. The term “substantially without” or “essentially without” as used herein means the specific material may be used in a manufacturing process in small amounts that do not materially affect the basic and novel characteristics of the compositions according to the disclosure. The term “substantially without” or “essentially without” as used herein may also mean that the specific material is not used in a manufacturing process but may still be present in a raw material that is included in the composition.

As used herein, the term “salts” refers to throughout the disclosure may include salts having a counter-ion such as an alkali metal, alkaline earth metal, or ammonium counterion. This list of counterions, however, is non-limiting.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “sulfate-based surfactant” as used herein, also means “sulfate-containing surfactant.” Thus, the term “essentially free of sulfate-based surfactant” also means “essentially free of sulfate-containing surfactant.”

As used herein, the term “surfactants,” as well as any specifically-identified surfactants, includes salts of the surfactants even if not explicitly stated.

As used herein, the term “surfactant system” refers to a combination of different surfactants. For example, the term “anionic surfactant system” refers to one anionic surfactant or a combination of different anionic surfactants, and the term “nonionic surfactant system” refers to one nonionic surfactant or a combination of different nonionic surfactants.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition.

### Example 1—Cleansing Compositions

The following inventive compositions were prepared according to the formulations set forth in Table 1 below.

TABLE 1

| | Inventive | | | |
|---|---|---|---|---|
| | Compositions | | | |
| INCI Name | 1A | 1B | 1C | 1D |
| DISODIUM | 4.00 | 4.00 | 5.04 | 4.00 |
| COCOAMPHODIACETATE | | | | |
| COCAMIDOPROPYL | 3.23 | 3.23 | 8.66 | 3.23 |
| BETAINE | | | | |
| COCO-BETAINE | 1.56 | 1.56 | — | 1.56 |
| CAPRYLYL/CAPRYL | 3.0 | 3.0 | 3.9 | 3.0 |
| GLUCOSIDE | | | | |
| SODIUM COCOYL | — | — | — | 2.0 |
| ISETHIONATE | | | | |
| SODIUM C14-16 OLEFIN | 0.87 | — | 2.66 | — |
| SULFONATE | | | | |
| SODIUM METHYL | — | 0.87 | — | — |
| COCOYL TAURATE | | | | |
| STEARAMIDOPROPYL | 2.5 | 2.5 | 2.0 | 2.5 |
| DIMETHYLAMINE | | | | |
| XANTHAN GUM | 0.42 | 0.42 | 0.45 | 0.42 |
| CITRIC ACID | 0.96 | 0.95 | 0.94 | 0.85 |
| ADDITIVES (fragrance, | 1.64 | 1.63 | 2.30 | 1.90 |
| preservatives, thickeners) | | | | |
| SOLVENT (water + non- | QS | QS | QS | QS |
| aqueous solvents) | | | | |

Each of Inventive Compositions 1A-1D was prepared by first mixing the solvents and polysaccharide thickener in a homogenizer for two minutes. The speed was then reduced and the temperature increased and the fatty amine was added. Once the fatty amine was thoroughly incorporated, the speed was increased and the disodium cocoamphodiacetate was added, and mixing continued for 15 minutes. The temperature was again reduced and the remaining ingredients were added in stages, until the final composition was homogenous.

Inventive Compositions 1A-1C were semi-transparent and stable. They had a pH of about 5.29, 5.42, and 5.4, respectively. Their viscosities were about 10 seconds, 10 seconds, and 36 seconds of Efflux time, respectively, when measured using Ford cup 8, corresponding to about the viscosity in Brookfield (spindle #4, 20 RPM) of 3700 cPs, 5100 cPs, and 16,500 cPs.

### Example 2—Foaming Analysis

A foaming study was conducted to analyze the foaming properties of Inventive Compositions 1A-1C of Table 1, in comparison with a commercially available shampoo that contained both sulfate-based surfactants (e.g., sodium laureth sulfate) and silicones (Comparative Composition C1).

The foaming analysis was performed on a foam analyzer as follows: a) prepare a 10% solution for each test composition sample by diluting the composition to 10% with tap water at 25° C.; b) vigorously agitate the 10% solution of each test composition sample using a high speed mixer for less than 15 seconds; c) add the solution into the cylinder of foam analyzer; d) stir solution for 15 seconds to generate foam; and e) after 15 seconds, stop stirring and monitor the height of generated foams, liquid height (i.e., liquid drainage from foams) in the bottom of the cylinder, and the total height of the foam and the liquid at the bottom. In addition, the structures of foam generated by Inventive Compositions 1A-1C and Comparative Composition C1 were analyzed.

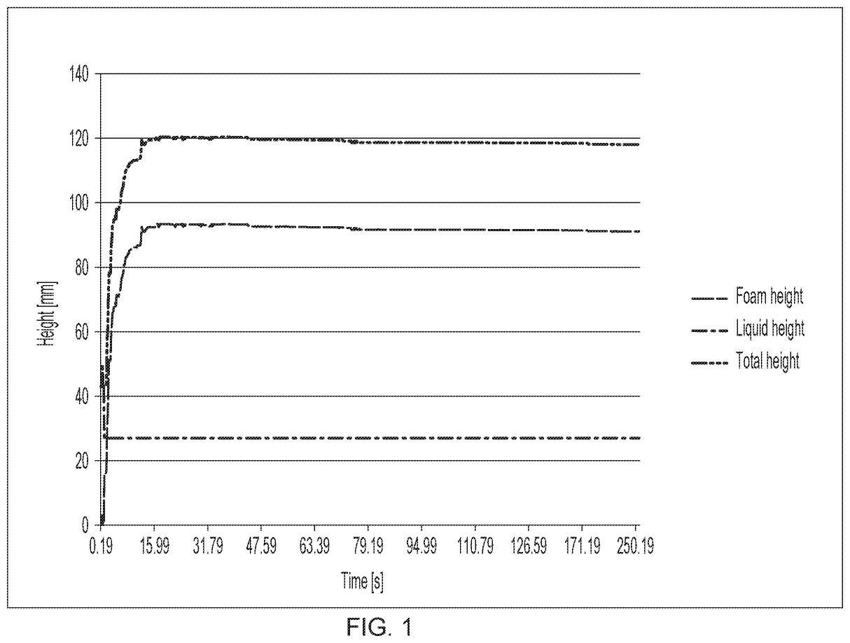

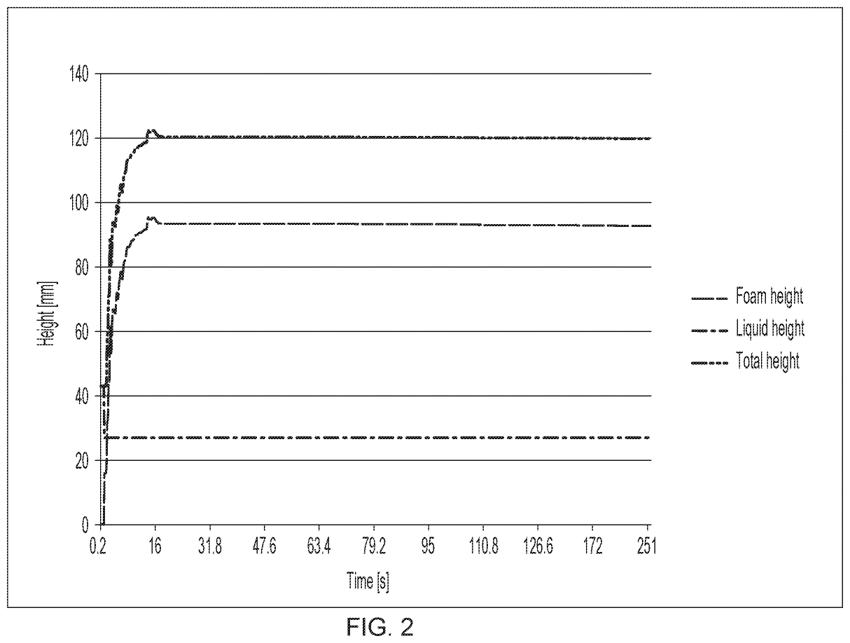

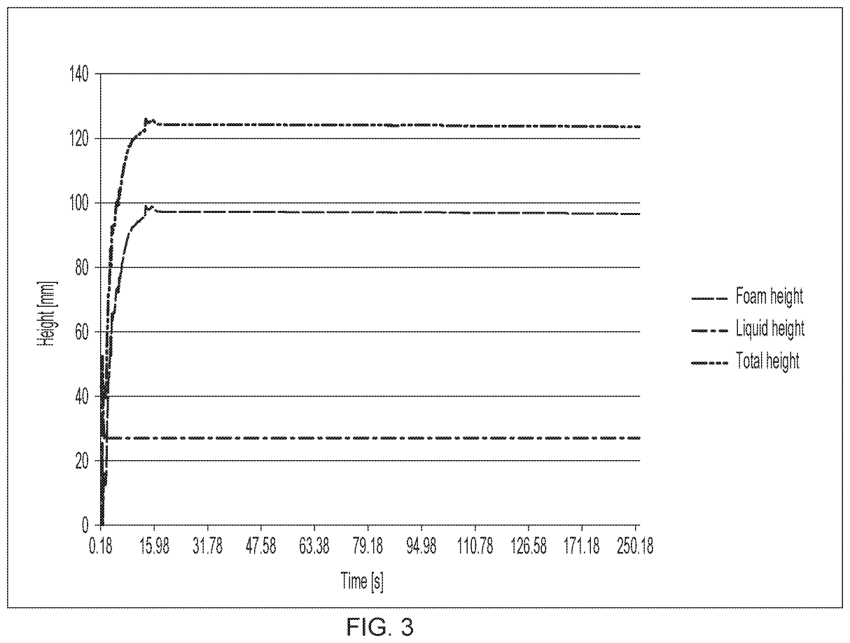

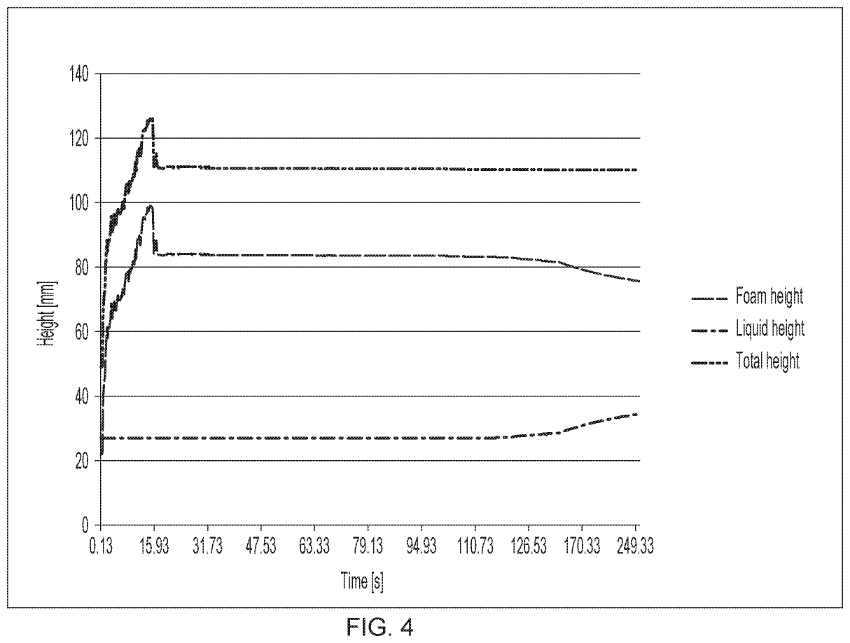

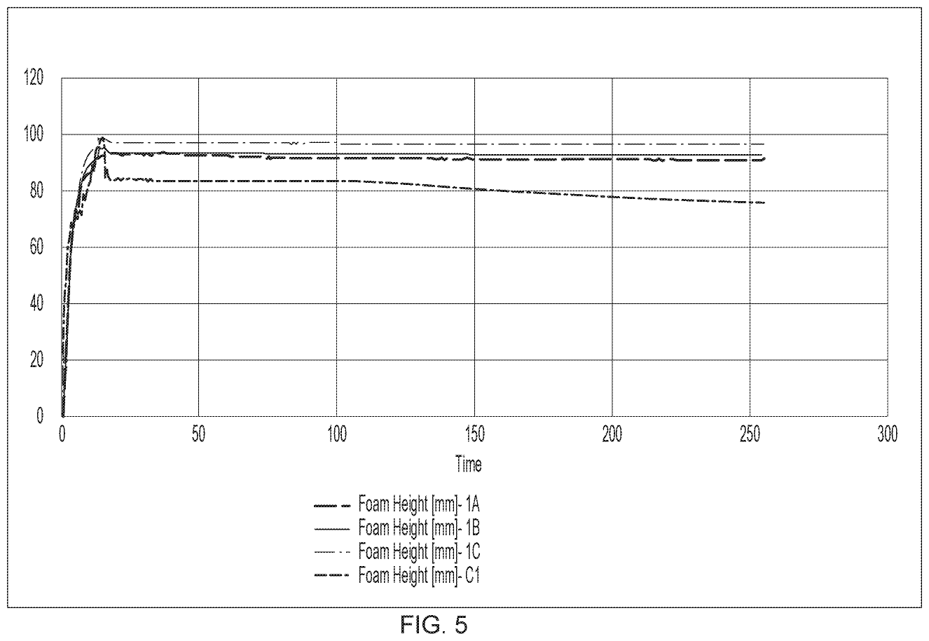

FIGS. 1-3 are graphs illustrating the foaming performance of Inventive Compositions 1A-1C measured during the foaming analysis. FIG. 4 is a graph illustrating the foaming performance of Comparative Composition C1 during the foaming analysis. FIG. 5 is a graph comparing the height of foams generated by Inventive Compositions 1A-1C and Comparative Composition C1 during a period of time. As shown in FIGS. 1-5, foams generated by all three Inventive Compositions 1A-1C reached a height between 90 mm and 100 mm at about 16 seconds, and kept at that height for more than about 250 seconds. In contrast, while the foam generated by Comparative Composition C1 reached to a height near 100 mm at about 16 seconds, it quickly decreased to a level near 80 mm within a few seconds. The foam of Comparative Composition C1 started to turn into liquid, with a foam height decreased below 80 mm within 160 seconds and its liquid level started to increase in correspondence. The liquid height of all three Inventive Compositions 1A-1C also kept at about the same level. The results showed that all three Inventive Compositions 1A-1C had improved foaming performance over Comparative Composition C1, such as improved flash foaming, foam abundance, and foam stability.

In addition, the structures of foams generated by Inventive Compositions 1A-1C and Comparative Composition C1 were studied. In this foam structure study, bubble number (bubble count/mm²) and mean bubble area (MBA) (bubble size) were measured. Structure data of the air bubble sizes and the change of the bubble size with time indicated that the foams formed by the Inventive Compositions were creamy and stable.

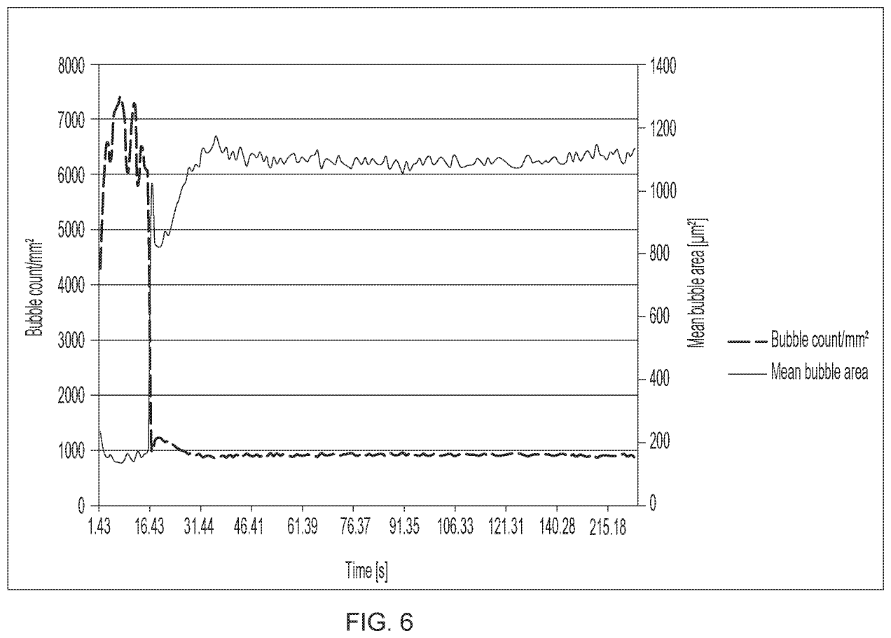

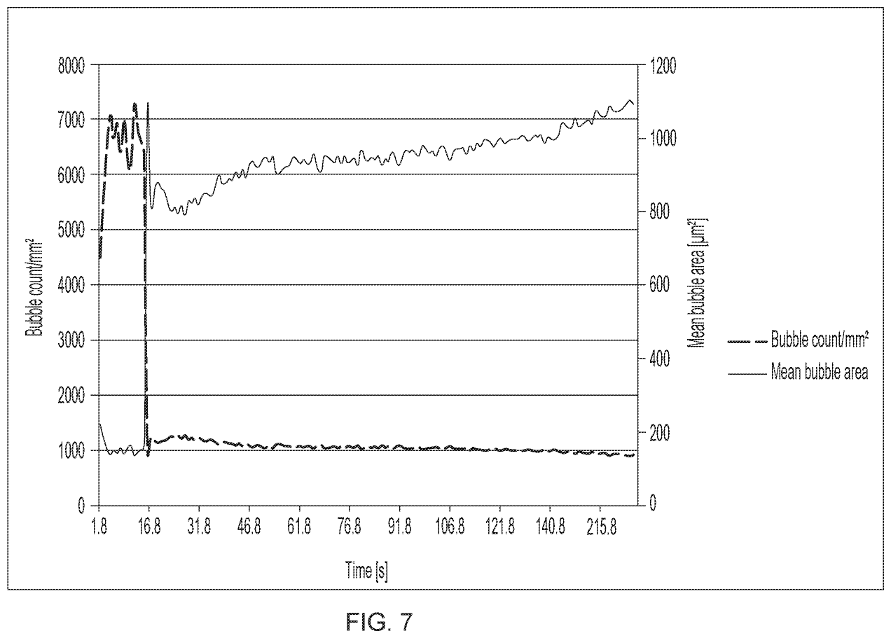

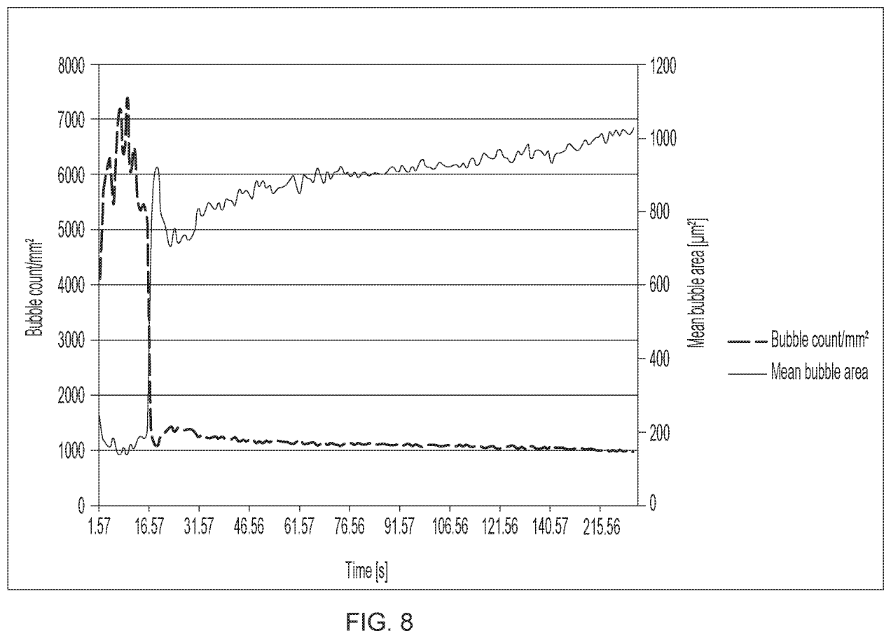

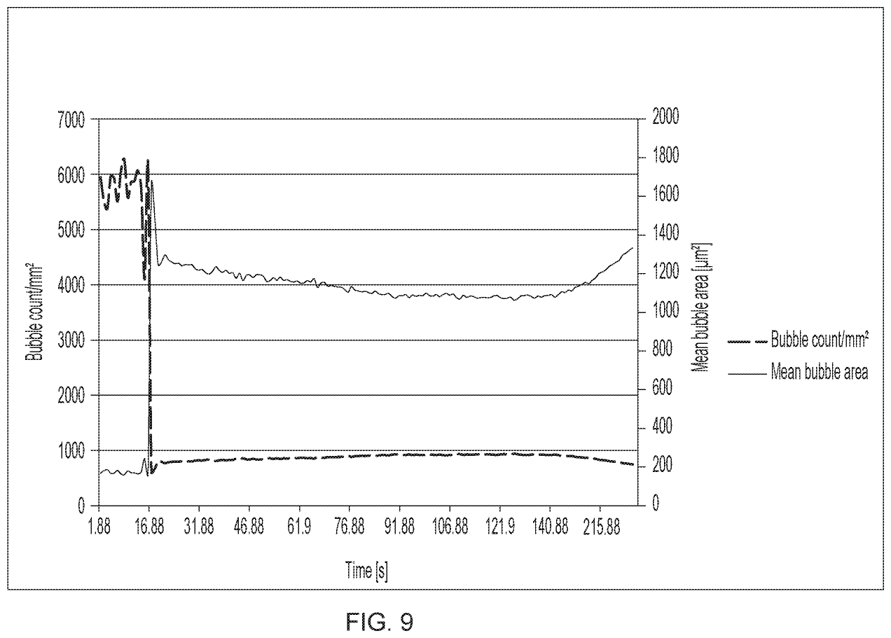

FIGS. 6-9 are graphs illustrating bubble count/mm² and mean bubble area (MBA) of the foams generated by compositions 1A-1C and C1. As shown in FIGS. 6-9, the bubble count/mm² of Compositions 1A-1C stays around 1000 over a prolonged period time, while that of Comparative Composition C1 is around 900. The Structure data shows that for Inventive Compositions 1A-1C, the MBA remains at either more than 1000 or increases to over 1000 over time. On the other hand, the MBA for the Comparative Composition C1 decreases over time then starts to increase at the end of the test period. The results showed that compared to the foams generated by Comparative Composition C1, the foams generated by compositions disclosed herein were surprisingly creamy and stable. Thus, the cleansing compositions according to the disclosure have excellent foaming properties despite the absence of sulfate-based surfactants.

### Example 3—Evaluation of Performance

A comparative study was conducted to evaluate the performance of Inventive Compositions 1A-1C, prepared according to the formulations of Table 1, when used as shampoos, in comparison with the commercially available shampoo used in Example 2, i.e., Comparative Composition C1. The study was performed on the hair of mannequin models, hair swatches, and individual volunteer's heads.

### Mannequin Test

In the mannequin test, long, straight, bleached, wet hair on one half of the head of mannequin models was treated with one of Inventive Composition 1A-1C, and the other half of the head was treated with Comparative Composition C1, side by side. About 5 grams of one of Inventive Composition 1A-1C was applied to one-half of head of a mannequin model's hair and was distributed evenly with fingers and lathered. The same amount of Comparative Composition C1 was then applied to the other half of the head of the mannequin model's hair and was distributed evenly with fingers and lathered. Both the Inventive Compositions and Comparative Composition were allowed to remain on the hair for about 2 minutes, followed by thorough rinsing. After rinsing the compositions from the hair, foaming quality & quantity and conditioning effects were evaluated.

As evaluated by the experts, the overall performance of each of Inventive Compositions 1A-1C was similar to that of Comparative Composition C1, without the disadvantages of a sulfate-based shampoo.

### Hair Swatch Test

In the hair swatch test, a medium bleached hair swatch was used. In particular, 0.5 gram of each sample of Inventive Compositions 1A-1C and Comparative Composition C1 was applied onto the wet hair swatch and distributed evenly with fingers, and was allowed to lather, and then rinsed off. After rinsing the compositions from the hair, the hair was combed while it was wet, dried, and styled. The hair swatch test was conducted to both wet and dry hair.

As evaluated by the experts, the overall performance of each of Inventive Compositions 1A-1C was similar to that of Comparative Composition C1, without the disadvantages of a sulfate-based shampoo.

### In Vivo Test

In the in vivo test, the inventive compositions were tested and compared to the comparative composition on 3 individual volunteer's heads using similar methods as applied to mannequin models.

As evaluated by the experts, the overall performance of each of Inventive Compositions 1A-1C was similar to that of Comparative Composition C1, without the disadvantages of a sulfate-based shampoo.

Based on the above Examples, it was demonstrated that compositions according to the disclosure provide excellent foaming and performance properties relative to compositions that are not free or essentially free of sulfate-based anionic surfactants.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1. A composition for cleansing keratin materials comprising:
(a) at least one first amphoteric surfactant chosen from alkyl amphoacetates, alkyl amphodiacetates, salts thereof, or mixtures thereof;
(b) at least one second amphoteric surfactant chosen from betaines or salts thereof;
(c) at least one nonionic surfactant;
(d) at least one non-sulfate anionic surfactant;
(e) at least one fatty amine; and
(f) at least one polysaccharide thickening agent;
wherein the composition is essentially free of sulfate-based surfactants.

2. The composition of claim 1, wherein the composition is essentially free of silicones.

3. The composition of claim 1, wherein the total amounts of the at least one first amphoteric surfactant and the at least one second amphoteric surfactant independently range from about 0.1% to about 15% by weight, relative to the total weight of the composition.

4. The composition of claim 1, wherein the total amount of the at least one first amphoteric surfactant and the at least one second amphoteric surfactant ranges from about 5% to about 20% by weight, relative to the total weight of the composition.

5. The composition of claim 1, wherein the total amount of amphoteric surfactants is greater than the combined amount of non-ionic and non-sulfate anionic surfactants.

6. The composition of claim 1, wherein the at least one first amphoteric surfactant comprises at least one (C8-C20)alkylamphodiacetate, and the at least one second amphoteric surfactant comprises at least one compound chosen from alkyl betaines, amido betaines, or mixtures thereof.

7. The composition of claim 1, wherein the weight ratio of the at least one first amphoteric surfactant to the at least one second amphoteric surfactant ranges from about 1:10 to about 10:1.

8. The composition of claim 1, wherein the at least one nonionic surfactant is chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides (“glucosides”) or polyglycosides, alkyl and polyalkyl esters of sucrose, optionally polyoxyethylenated alkyl and polyalkyl esters of glycerol, and optionally polyoxyethylenated alkyl and polyalkyl ethers of glycerol.

9. The composition of claim 1, wherein the at least one nonionic surfactant comprises at least one alkylpolyglucoside.

10. The composition of claim 1, wherein the at least one nonionic surfactant is present in an amount ranging from about 0.1% to about 10% by weight, relative to the total weight of the composition.

11. The composition of claim 1, wherein the at least one non-sulfate anionic surfactant is chosen from alkyl sulfonates, alkyl sulfosuccinates, alkyl sulfoacetates, acyl isethionates, alkoxylated monoacids, acyl amino acids, acyl glycinates, acyl glutamates, acyl sarcosinates, salts thereof, or mixtures thereof.

12. The composition of claim 1, wherein the at least one non-sulfate anionic surfactant is chosen from alkyl sulfonates, acyl isethionates, acyl taurates, salts thereof, or mixtures thereof.

13. The composition of claim 1, wherein the at least one non-sulfate anionic surfactant is present in an amount ranging from about 0.01% to about 6% by weight, relative to the total weight of the composition.

14. The composition of claim 1, wherein the at least one fatty amine is present in an amount ranging from about 0.01% to about 10% by weight, relative to the total weight of the composition.

15. The composition of claim 1, wherein the at least one polysaccharide thickening agent is chosen from gums, celluloses, and starches.

16. The composition of claim 1, wherein the at least one polysaccharide thickening agent is present in an amount ranging from about 0.01% to about 5% by weight, relative to the total weight of the composition.

17. The composition of claim 1, wherein the composition is essentially free of cationic polymers.

18. A composition for cleansing keratin materials comprising:
(a) at least one first amphoteric surfactant chosen (C8-C20)alkyl-amphodiacetates or salts thereof;
(b) at least one second amphoteric surfactant chosen from alkyl betaines, amido betaines, salts thereof, or mixtures thereof;
(c) at least one nonionic surfactant chosen from alkylpolyglucosides;
(d) at least one non-sulfate anionic surfactant chosen from alkyl sulfonates, alkyl sulfosuccinates, acyl isethionates, acyl taurates, salts thereof, or mixtures thereof;
(e) at least one fatty amine; and
(f) at least one polysaccharide thickening agent chosen from gums;
wherein the composition is essentially free of sulfate-based surfactants, and
wherein the composition is essentially free of silicones.

19. A method for cleansing keratin materials comprising:
(i) applying to the keratin material a composition comprising:
(a) at least one first amphoteric surfactant chosen from alkyl amphoacetates, alkyl amphodiacetates, salts thereof, or mixtures thereof;
(b) at least one second amphoteric surfactant chosen from betaines or salts thereof;
(c) at least one nonionic surfactant;
(d) at least one non-sulfate anionic surfactant;
(e) at least one fatty amine; and
(f) at least one polysaccharide thickening agent;
wherein the composition is essentially free of sulfate-based surfactants; and
(ii) subsequently rinsing the composition from the keratin material.

20. The method of claim 19, wherein the keratin material is hair.
